(19) United States
(12) Patent Application Publication
(10) Pub. No.: US 2024/0013921 A1
(43) Pub. Date: Jan. 11, 2024

(54) GENERALIZED COMPUTATIONAL FRAMEWORK AND SYSTEM FOR INTEGRATIVE PREDICTION OF BIOMARKERS

(71) Applicant: INSYBIO INC., AUSTIN, TX (US)

(72) Inventors: KONSTANTINOS THEOFILATOS, PATRA (GR); CHRISTOS ALEXAKOS, PATRA (GR); AIGLI KORFIATI, PATRA (GR); CHRISTOS DIMITRAKOPOULOS, BASEL (CH); SEFERINA MAVROUDI, PATRA (GR)

(73) Assignee: INSYBIO INC., AUSTIN, TX (US)

(21) Appl. No.: 18/373,047

(22) Filed: Sep. 26, 2023

Related U.S. Application Data
(63) Continuation of application No. 15/837,407, filed on Dec. 11, 2017.
(60) Provisional application No. 62/432,981, filed on Dec. 12, 2016.

(51) Int. Cl. G16H 50/20; G16H 50/50; G16B 20/00; G16B 30/00; G16B 40/00; G16H 20/10; G16B 40/20; G16B 30/10; G16B 20/20

(52) U.S. Cl. CPC G16H 50/20; G16H 50/50; G16B 20/00; G16B 30/00; G16B 40/00; G16H 20/10; G16B 40/20; G16B 30/10; G16B 20/20; G06N 7/01

(57) ABSTRACT

An approach is provided to computationally identify biomarkers associated with diseases and medical conditions. The procedure first identifies biomarkers individually at the DNA, RNA and proteome levels. Then provides a methodology to integrate the single-source biomarkers and perform dimensionality reduction in order to detect the most informative subset of biomarkers that better distinguish samples between two biological conditions (disease vs normal samples). The dimensionality reduction step minimizing biases due to unnecessary or partially correlated biomarkers and significantly reduces the search space of possible biomarkers. An algorithm is also described for the automated optimization of the proposed DNA-seq and RNA-seq pipelines.

### RELATED APPLICATIONS

This application is a continuation of U.S. Non-Provisional application Ser. No. 15/837,407, filed on Dec. 11, 2017, which claims the benefit of U.S. Provisional Application No. 62/432,981, filed on Dec. 12, 2016, entitled “A GENERALIZED COMPUTATIONAL FRAMEOWRK AND SYSTEM FOR INTEGRATIVE POTENTIAL BIOMARKER DISCOVERY ANALYSIS”, commonly owned and assigned to the same assignee hereof.

### BACKGROUND

### Field

The present invention relates to the computational prediction of biomarkers by integrating data from various biological experiments.

### Background

Recent advances in genetics have helped the biological and medical community to explore the cause of diseases due to heredity factors or factors acquired during the lifetime of individuals. This quest for the causes of diseases has focused on the analysis of genes and other biological molecules. Such molecules, termed biomarkers, can be described as features that are objectively measured and evaluated as an indicator of normal biological processes, pathogenic processes (e.g. a disease or medical condition), or pharmacological responses to a therapeutic intervention (e.g. drug or other type of treatment).

During the last decades, the advances in the genomics, transcriptomics and proteomics experiments have resulted in discovering molecular biomarkers (e.g. proteins, RNAs, genes) and in exploring their pathogenic role. The role of molecular biomarkers has been studied by the research community in the prognosis, diagnosis and progression of diseases as well as in drug targeting and the prediction of drug response. However, existing experimental techniques are time-consuming and cost-inefficient in detecting disease-related biomarkers.

Existing techniques for the computational prediction of molecular biomarkers are mainly based on i) genomics technologies (e.g. DNA-sequencing), which identify genetic variants as biomarkers, ii) transcriptomics technologies (e.g. microarrays and RNA-sequencing), which identify transcripts with significantly altered expression profiles between two biological conditions and iii) proteomics technologies (e.g. mass spectrometry), which uncover biomarkers at the protein and/or peptide level.

The computational prediction of biomarkers uses genetic experimental data and applies statistics, clustering, optimization and other types of algorithms to identify correlations between seemingly unrelated data and uncover biomarkers that cannot be easily detected by experimental techniques. The current state-of-the-art on the computational prediction of biomarkers is mostly focused on tools and methods, which use only one type of data (genomics, transcriptomics, proteomics etc.). Some other methods try to combine different types of data in order to improve the task of predicting biomarkers.

Because of the vast amount of information (i.e. high-throughput experimental data) that needs to be taken into account in the computational analysis and the very few samples available (relatively speaking), methods for the computational prediction of biomarkers fail to find solutions that provide significant improvements in specific diseases or medical conditions or even in the use of general purpose.

### SUMMARY

The current invention provides an approach to computationally predict biological molecules as biomarkers associated with diseases and medical conditions. Biomarker prediction is performed on disparate omics data by mixing various types of algorithms, including clustering, feature selection and optimization. The proposed methodology exhibits high accuracy in predicting biomarkers and minimizes bias due to unnecessary or partially correlated inputs that could result in false predictions.

The proposed approach consists of an improved RNA sequencing analysis that exploits non-coding RNA, short RNA reads, and unassigned RNA reads to improve accuracy of the prediction of biomarkers at the RNA level.

Finally, the current invention proposes an automated solution for optimizing the ordering of the algorithmic steps and their internal parameters.

### BRIEF DESCRIPTION OF THE DRAWINGS

FIG. 1 shows system 100 implementing the present innovative solution.

FIG. 2 shows the architecture of a computing device.

FIG. 3 shows the main software components of a device or apparatus.

FIG. 4 shows the main software components of a server.

FIG. 5 is a flowchart showing the main steps performed to predict biomarkers using different types of biological data.

FIG. 6 shows the main steps of a genetic algorithm.

FIG. 7 is a flowchart showing the main steps performed to identify potential biomarkers at the DNA level.

FIG. 8 is a flowchart showing the main steps performed to identify potential biomarkers at the RNA level.

FIG. 9 is a flowchart showing the main steps performed to automate the optimization of the steps of algorithms used for biomarker discovery in specific diseases and medical conditions.

FIG. 10 shows an example of an integrative biological network.

FIG. 11 shows an example of a clustered integrative biological network.

FIG. 12 shows an example of the application of the steps 640, 650.

FIG. 13 shows an example quality score for each read position in the .fastq RNA-sequencing data files.

### DETAILED DESCRIPTION

The word “exemplary” is used herein to mean “serving as an example, instance, or illustration”. Any embodiment described herein as “exemplary” is not necessarily to be construed as preferred or advantageous over other embodiments.

The terms “cellular” and intercellular” may be used interchangeably where combined with the word “component” or its plural form and refer to the same element(s).

The acronym “GO” is intended to mean “Gene Ontology”.

The term “mobile device” may be used interchangeably with “client device” and “device with wireless capabilities”.

The following terms have the following meanings when used herein and in the appended claims. Terms not specifically defined herein have their art recognized meaning.

An “amino acid” is a molecule having the structure wherein a central carbon atom (the α-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue”.

DNA (Deoxyribonucleic acid) is a molecule that carries the genetic instructions used in the growth, development, functioning and reproduction of all known living organisms.

A gene mutation or variant is an alteration in the DNA sequence that makes up a gene, such that the gene sequence differs from what is usually found in same type tissues. The most common types of mutations are Single Nucleotide Polymorphisms (SNPs) which are defined as the alternation of only one nucleic acid in a gene. Other known types of mutations are insertions, which are defined as the insertion of a nucleic acid sequence in a specific point of a gene, and deletions, which are defined as the removal of a part of a gene.

Essential genes are the ones for which normal functioning is vital for the survival of the cell. If one of the essential genes is not present or is not functioning properly, the cell cannot survive.

RNA (Ribonucleic acid) is a nucleic acid molecule similar to DNA but containing ribose rather than deoxyribose. RNA is formed upon a DNA template.

A noncoding RNA (ncRNA) is a functional RNA molecule that is transcribed from DNA but not translated into protein.

Protein refers to any polymer of two or more individual amino acids (whether or not naturally occurring) linked via a peptide bond, and occurs when the carboxyl carbon atom of the carboxylic acid group bonded to the α-carbon of one amino acid (or amino acid residue) becomes covalently bound to the amino nitrogen atom of amino group bonded to the α-carbon of an adjacent amino acid. The term “protein” is understood to include the terms “polypeptide” and “peptide” (which, at times may be used interchangeably herein) within its meaning. In addition, proteins comprising multiple polypeptide subunits (e.g., DNA polymerase III, RNA polymerase II) or other components (for example, an RNA molecule, as occurs in telomerase) will also be understood to be included within the meaning of “protein” as used herein. Similarly, fragments of proteins and polypeptides are also within the scope of the invention and may be referred to herein as “proteins”.

Protein-protein interactions (PPIs) are defined as functional or physical interactions between two proteins.

Biological network is defined as a graph-based representation of biological molecules and their interactions. In specific, nodes in this network are biological molecules such as proteins, genes, RNA etc., while edges are added between two nodes if there exist a known functional or physical interaction between the two nodes.

As used herein and in the appended claims, the singular forms “a,” “and,” and “the” include plural referents unless the context clearly dictates otherwise. Thus, for example, reference to “a biomarker” includes a plurality of biomarkers and reference to “biological networks” generally includes reference to one or more biological networks and equivalents thereof known to those skilled in bioinformatics and/or molecular biology.

Unless defined otherwise, all technical and scientific terms used herein have the same meaning as commonly understood to one of ordinary skill in the art to which this invention belongs (systems biology, bioinformatics). Although any methods similar or equivalent to those described herein can be used in the practice or testing of the invention, the preferred methods are described.

All publications mentioned herein are incorporated by reference in full for the purpose of describing and disclosing the databases, proteins, and methodologies, which are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The invention can be implemented either as a method, a software program implementing the method, or as a microprocessor, or a computer, or a computing device, apparatus or analyzer. The description of the invention is presented, for simplicity, in terms of the method implementing it but it is assumed to equally apply to the other forms of implementation previously mentioned.

Computational discovery of molecular biomarkers is mainly based on i) genomics technologies, such as DNA-sequencing, which identify variants as biomarkers (i.e. genes differing from their corresponding “normal” genes in the DNA sequence), ii) transcriptomics technologies, such as microarrays and RNA-sequencing, which identify transcripts (i.e. the single-stranded RNA product synthesized by transcription of DNA) with significantly altered expression profiles between two biological conditions and iii) proteomics technologies, such as mass spectrometry, which uncover biomarkers in a peptide and/or protein level.

The proposed innovative solution to computational biomarker discovery targets the problems of prior art approaches, namely the scarcity of experimental samples for the vast number of biological molecules that need to be analyzed. In addition to this, the present innovative solution proposes a novel computational analysis solution that simplifies the analysis process and suits the capabilities and needs of biologists and doctors who lack the technical skill and understanding, and bioinformaticians who do not master biomedical concepts in depth.

The innovative nature of the proposed solution lies in (i) the use of a wide variety of available data, far wider than any known prior art technique, by appropriate handling and integrating disparate data from distributed sources, (ii) the use of existing mathematical algorithms in a novel way by first combining optimized “pipelines” of multiple algorithms executed serially and in parallel, and then reducing dimensionality in order to minimize bias caused by data conveying no new information to the analysis, (iii) the automated optimization of the algorithmic parameters and order of their execution in specific diseases and medical conditions, and (iv) the use of non-coding RNA in biomarker identification.

The proposed innovative solution bypasses the shortcomings of prior art by using existing biological knowledge to guide the feature selection process in the input data. This is not trivial because there is a knowledge gap between machine learning experts and biologists. Moreover, even machine learning experts are mostly dealing with specific types of data and the integration of different types of omics is still an open field. The proposal described below exploits additional data such as Gene Ontology (GO) terms, clinical data, microarray experiments, and goes into different levels of transcriptomics analysis by using non-coding RNA and short reads in addition to standard RNA.

The innovative nature of the proposed solution is also proven by the lack of commercial products that can handle such a wide range of disparate data and use them to guide the execution of their algorithmic solutions. The reason for this luck of commercial products can be attributed to the fact that bioinformatics analyses are prone to bias towards the big number of options researchers have to choose regarding algorithms, order of execution and parameter selection for each step and for each disease. There does not exist a universally good solution, thereby not a product that can be used by biologists and doctors to cover their needs, that is highly accurate, fast and cost-effective. The proposed solution not only presents improvements to prior art and new solutions to fill research and commercial product gaps but also provides an automation of the proposed innovations to optimize such a computation. As a result, the innovative product can be marketed not only for its accuracy, efficiency and usability improvement but also as a cheaper product (or service) that can cover scientific and commercial needs and significantly reduce time of the analyses.

Main Challenges Addressed by the Proposed Innovative Solution

The main challenge addressed by the proposed innovative solution is to reduce bias in the final output (i.e. list of annotated biomarkers) from the wide range of disparate input data and the parameters and order of execution of the chosen algorithms. This is achieved by selecting the available features using optimization techniques to guide parameter selections for the executed algorithms.

Furthermore, the challenge of optimizing the parameters and order of execution of the chosen algorithms is an almost impossible task for a user as the number of options and combinations for each disease and medical conditions that need to be tested is astronomical. This challenge is further aggravated as new algorithms are continuously taught in art that can be used in the individual steps of the proposed innovative solution. This situation renders the proposed solution not a simple automation of a manual routine that can be executed by a scientist or an engineer. Instead, the present solution is the only practical, efficient, and cost-effective solution to the problem at hand and the one not introducing any human bias or error.

Description of the Proposed Innovative Solution

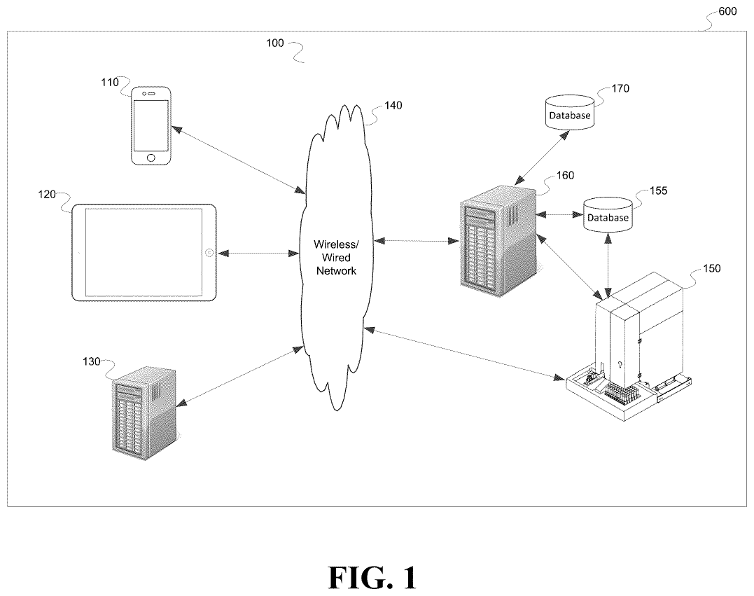

FIG. 1 shows system 100 implementing the present innovative solution. The system comprises main computing infrastructure 160 (physical, virtual, or cloud server), one or more user devices (smart phone 110, tablet 120, desktop or laptop computer 130), databases 170 (public or private), microarray analysis apparatus (150), and data database or other local storage 155. The components of system 100 are connected to each other via private or public networks, comprising wired and wireless networks, cloud-based communication or other similar data communications infrastructure.

The present innovative solution is executed at main computing infrastructure 160 or at a distributed computing infrastructure (e.g. of the type used in cloud computing or other distributed computing system paradigms—not shown in FIG. 1). In a variation of this exemplary system embodiment, the present innovative solution can be implemented at any computing infrastructure or distributed infrastructure, including the user's device or devices. For simplicity, the following disclosure and example of the present invention is done using the main computing infrastructure 160 as the place where the present innovative solution is executed.

A user may use mobile phone 110, or tablet 120, or networked desktop or laptop computer 130 and access, server 160, via wired or wireless network 140, which server provides access to public and/private databases 170. Such databases store experimental and computational data in the fields of genomics, transcriptomics, proteomics, GO, clinical data, etc. The user can view such data on his user device 110, 120, 130 and he may interact with the main computing infrastructure 160 to guide operation of the present innovative solution and view the final biomarkers and associated information produced by the innovative solution.

The user's devices and the server 160 also have access to biological data analyzer unit 150 (e.g. a microarray analyzer), which analyzer unit 150 provides experimental results on the microarray data. The biological data analyzer unit 150 stores its data either directly at the server 160 local storage, or at database 155.

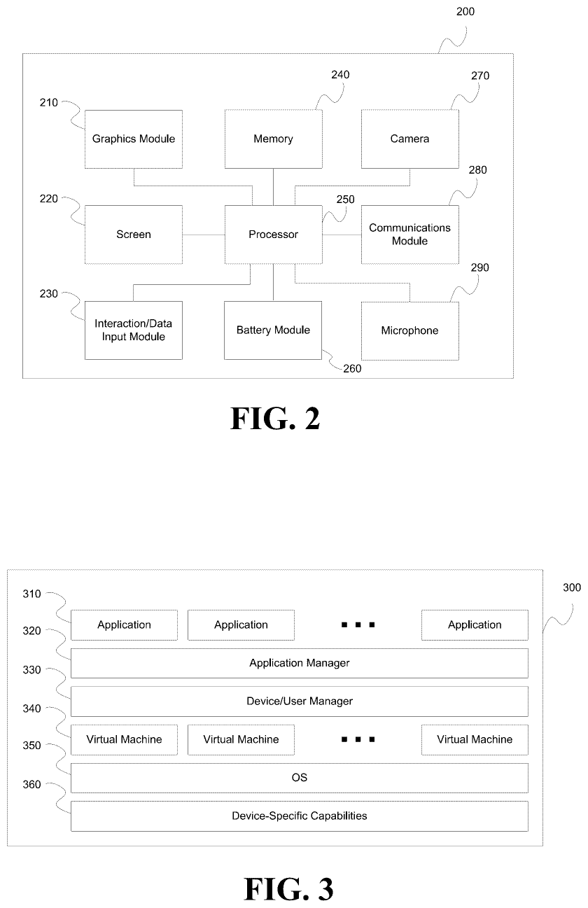

FIG. 2 shows the architecture of a computing device. Such computing device 200 comprises user devices 110, 120, 130, server 160, and biological analyzer 150, which implement the present innovative solution or part or parts of the innovative solution. Device 200 comprises Processor 250 upon which Graphics Module 210, Screen 220 (in some exemplary embodiments the screen may be omitted), Interaction/Data Input Module 230, Memory 240, Battery Module 260 (in some exemplary embodiments the battery module may be omitted), Camera 270 (in some exemplary embodiments the screen may be omitted), Communications Module 280, and Microphone 290 (in some exemplary embodiments the microphone may be omitted).

FIG. 3 shows the main Software Components of a device or apparatus. At the lowest layer of software components 300 are Device-Specific Capabilities 360, that is the device-specific commands for controlling the various device hardware components. Moving to higher layers lie OS 350, Virtual Machines 340 (like a Java Virtual Machine), Device/User Manager 330, Application Manager 320, and at the top layer, Applications 310. These applications may access, manipulate and display data.

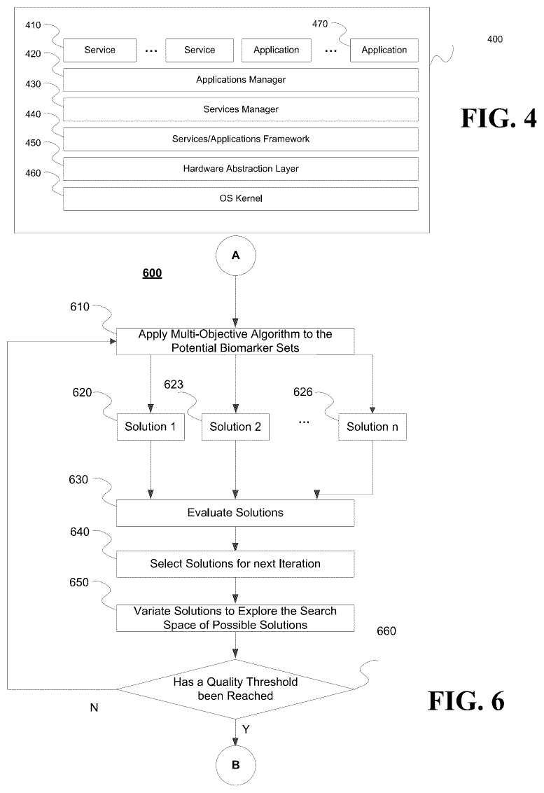

FIG. 4 shows the main Software Components of a Server. At the lowest layer of the software components 400 is OS Kernel 460 followed by Hardware Abstraction Layer 450, Services/Applications Framework 440, Services Manager 430, Applications Manager 420, and Services 410 and Applications 470.

It is noted, that the software and hardware components shown in FIG. 2, FIG. 3 and FIG. 4 are by means of example and other components may be present but not shown in these figures, or some of the displayed components may be omitted.

The present innovative solution can also be implemented by software written in any programming language, or in an abstract language (e.g. a metadata-based description which is then interpreted by a software or hardware component). The software running in the above-mentioned hardware, effectively transforms a general-purpose or a special-purpose hardware or computing device, apparatus or system into one that specifically implements the present innovative solution.

Alternatively, the present innovative solution can be implemented in ASIC or other hardware technology.

Despite the promising results of the prior art for biomarker discovery in the genome and transcriptome levels only a few approaches combine more than two types of experiments in an integrated biomarker discovery solution. In addition, most of them are based on simple statistical and/or dimensionality reduction techniques to capture the underlying biological mechanisms. A pipeline for biomarker discovery has been described in prior art that combines different data types; however, the integration of the different data is only accomplished by computing the significance of the correlation between pairs of the data types. In another prior art teaching, a network-based method is presented for the discovery of biomarkers, but it takes into account only DNA-sequencing data in the form of single nucleotide polymorphisms. A more general integration approach analyses in RNA-Seq, proteomics, metabolomics and lipidomics data are analyzed sequentially. The molecules that are found differentially expressed in one experiment narrow down the inputs of the next analysis emphasizing only on the molecules, which are their biological products. A more general idea is to combine transcriptomics and proteomics data to uncover molecules, which are significantly differentially expressed in both types of data in order to remove false positives. However, this approach does not take into account differentiations that occur at the level of post-translational modifications. In addition, the level on which one measures the differential expression depends on the type of molecule. For example, the protein level of a transcription factor is more informative than its RNA level whereas a kinase's phosphoproteome level is more informative than its RNA level. Therefore, the careful integration of data from different cellular molecules is essential for identifying biomarkers.

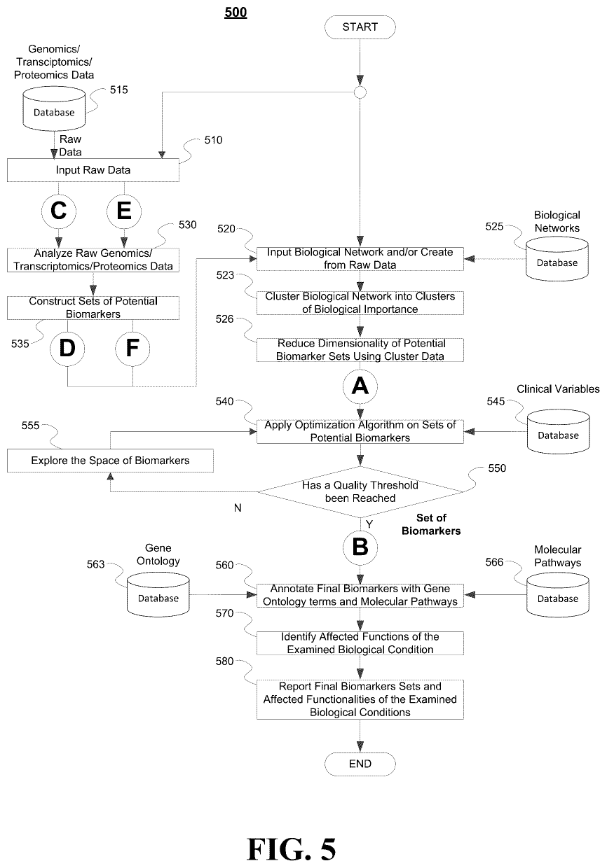

The series of steps presented in FIG. 5 solve the above shortcomings of prior art and also solve the problem of combining various types of data for biomarker discovery.

FIG. 5 is a flowchart showing the main steps performed to predict using different types of biological data. FIG. 5 processing steps 500 may be replaced by other similar steps (e.g. substitution of an algorithm with another algorithm of the same type) and their order may be altered in alternative exemplary embodiments.

FIG. 5 processing integrates various biological data in order to increase accuracy of biomarker prediction, as well as, to identify biomarkers that are missed by prior art teachings. The different types of biological data used in the following processing steps are produced by experimentally analyzing the same (physical) biological samples.

The processing commences with the input of raw (unprocessed or pre-processed) data 510 from database(s) 515. These are different omics data measured in disease and their matched normal samples and comprise genomics (i.e. DNA), transcriptomics (i.e. mRNA, non-coding RNA, etc.) and proteomics (i.e. proteome and phosphoproteome) data etc. These data are typically available from public or private biological databases and are analyzed by steps C and E to predict biomarkers separately at the levels of DNA, RNA and proteome.

Processing continues at step 520, where biological networks are input from public or private databases 525 such as Biogrid, String, KEGG, Reactome, etc.

Such biological networks contain nodes and edges linking these nodes; edges indicate a relationship between the connected nodes. Every node of the network is a molecule (gene or protein) and every edge represents an interaction. The interactions are of different types and occur in different functional levels of the cell such as activation and inhibition between proteins or transcription factor binding to a target gene and enabling its expression.

Since experimental data (or computational approaches if such a network is created or processed computationally) may leave uncertainty as to the validity of the linking of the edges, a weighting of the edge may be used to show the related certainty.

Examples of biological networks can be found in public databases; however, there is a gap, as there are very few or no integrative biological networks that integrate multi-omics biological data. Such integrative networks can be created in step 520 by using available individual biological networks from database 525 and by integrating them. This can be done by scoring the interactions based on the number of databases that they are reported. By taking an analogy as example, one could consider that each individual network contains overlapping fragments of a sentence. The final integrative network contains different types of interactions such as, expression/repression at the RNA level, activation/inhibition at the protein level, phosphorylation/dephosphorylation at the phosphoproteome level. The integrative network is merged with data from D and F, which are the predicted biomarkers from the DNA, RNA and proteome levels 520. The merging is performed by mapping the biomarkers into biological network 520. The predicted biomarkers from D and F are used as a label for the network nodes. For example, a gene that is downregulated due to an inactivating mutation leads to the downregulation of other genes.

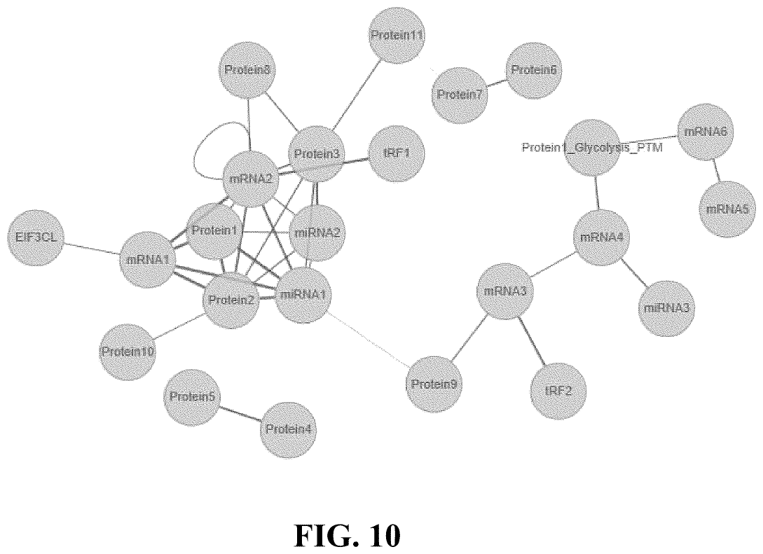

Continuing with the previous analogy, we may know that e.g. a protein is related to a gene, which is associated with a mutation, which mutation is a biomarker for a disease. Using this information we may deduce which mutations (i.e. mutated genes) are linked to the gene the mutations are associated with, non-coding RNAs are linked to the RNA whose expression they regulate, mRNAs are linked to the genes which genes are transcribed to the mRNAs and to the proteins the mRNAs are expressed to, proteins are linked to their peptides, genes are connected with proteins which are the genes' transcription factors, and proteins are linked to the proteins with which the proteins physically interact. In another embodiment, every edge in this integrative biological network has a weight, which reflects the confidence of this interaction. An example integrative biological network is shown in FIG. 10.

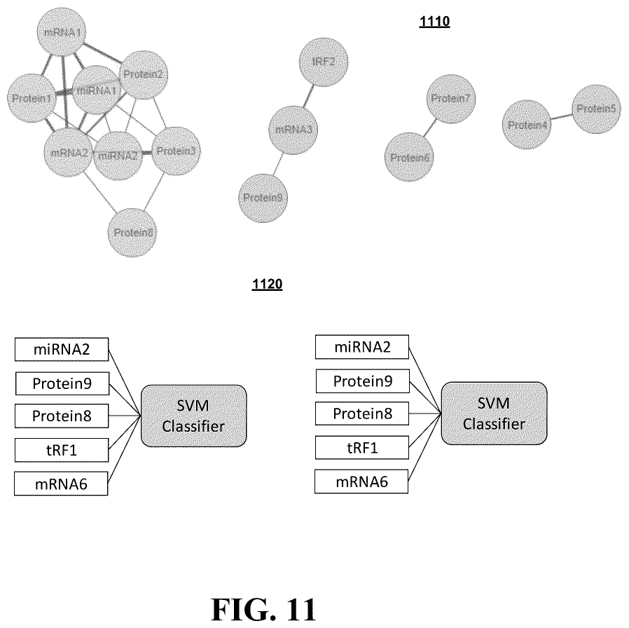

The next step (523) focuses on clustering the integrative biological network to uncover functional modules of biological importance. For step 523, an algorithm similar to ClusterONE or GENA is used which handles weighted networks and allow overlapping clusters. These algorithms can detect functional modules as groups of molecules that are strongly connected in the network and sparsely connected to the rest of the molecules in the network. These algorithms are given by means of example and do not limit the scope of the present innovative solution. It is possible to use any clustering algorithm. The clusters generated from this step are most likely associated with a known or unknown biological function. For example, the gene that is expressed in specific transcripts and/or mRNA and the protein which is then produced together with the related transcription factors, the non-coding RNAs which are regulating these mRNAs and the mutations of these genes are clustered together. An example of a clustered biological network is shown in FIG. 11.

The output of step 523 is clusters of biological molecules (genes, proteins etc.) that will be used as potential biomarkers.

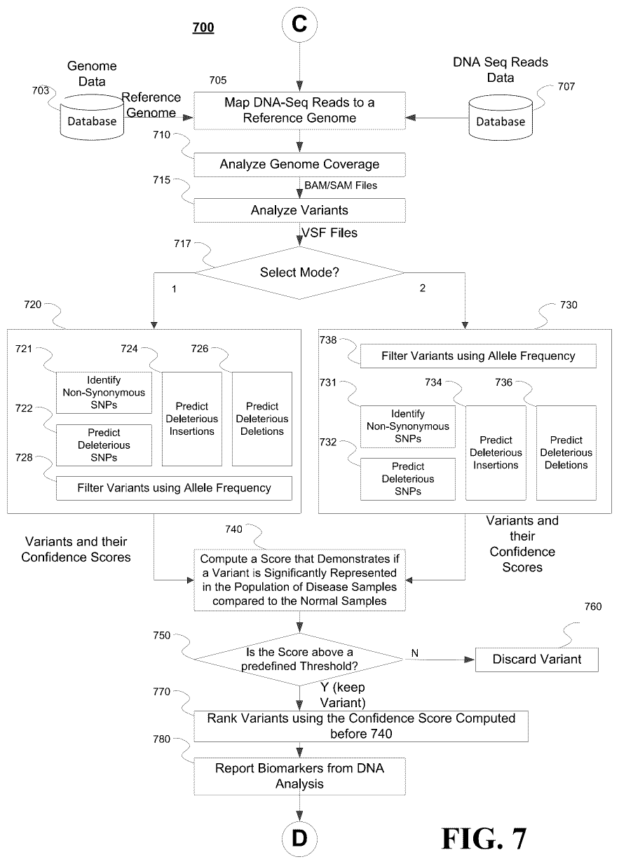

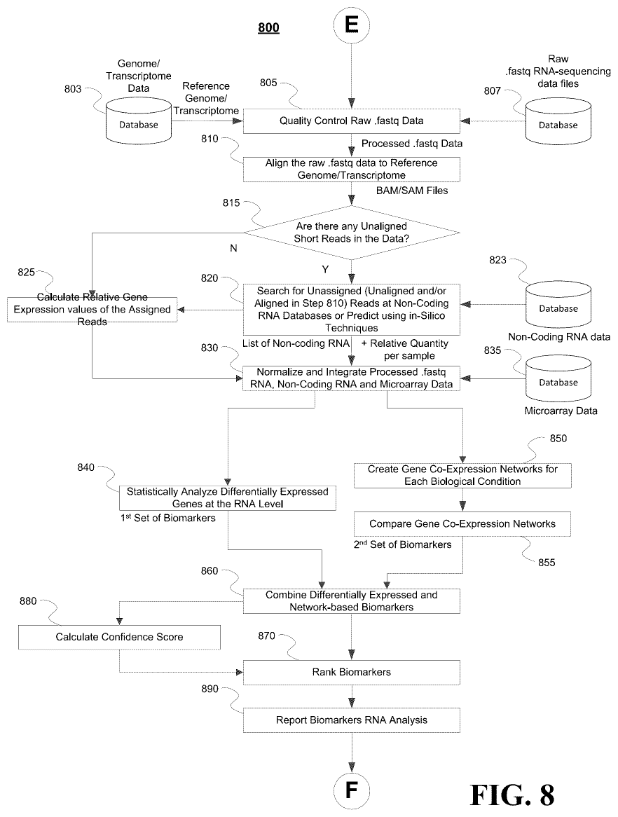

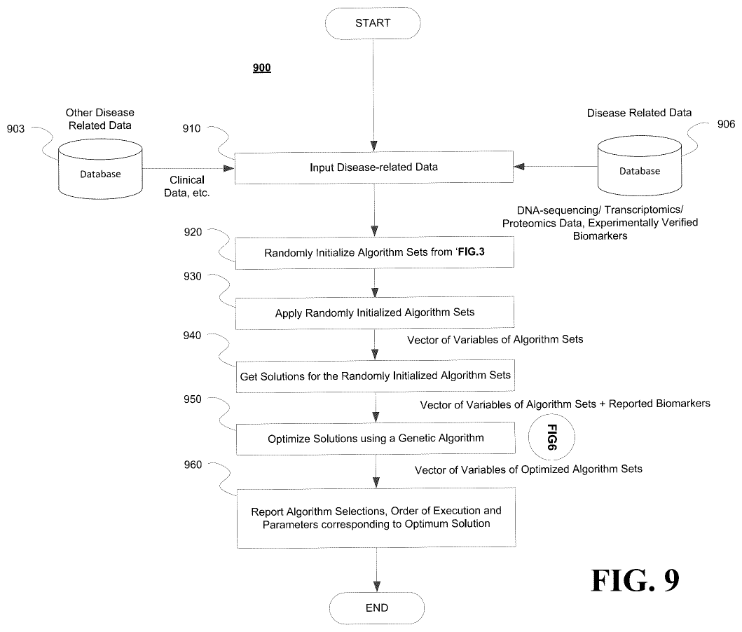

A processing is done to analyze the raw genomics, transcriptomics and proteomics data (step 530) and construct sets of potential biomarkers 535. Steps 530, 535 are executed in parallel with the construction (step 520). The steps 530 and 535 may be implemented by any analysis method of choice. Example of preferable methods (points C-D and E-F) are shown in FIG. 7 and FIG. 8, which methods produce as output biomarkers from DNA and RNA data analysis, respectively.

Proteomics data are being produced by analyzing bio fluids or samples from tissues using Mass Spectrometry based experimental instrumentation. Proteomics are analyzed with a similar technique, one of which is the “Quantify then Identify” technique. More information is given in the “Identifying Transcript Quantities as Biomarkers from Proteomics Data” section later in this description.

The clustered integrative biological networks and associated potential biomarkers from step 523 are fed as input to the step 526.

Step 526 uses the inputs from step 523 to reduce the dimensionality of the biomarkers from step 535 by using an optimization algorithm 540.

The importance of reducing dimensionality of the biomarker optimization problem goes well beyond the mere reduction of computational complexity and the increased calculation speed. Dimensionality reduction gives results that are more accurate and avoids bias introduced by the manual operation of the processing steps.

A vector represents each biomarker, which vector is a feature that will later be used as an input in a classifier. This vector is equal to the length of the available samples (disease and healthy). For example, every mRNA biomarker will have a relative expression measurement for each of the samples in this vector. The same holds for any other data source. Abundance measurements for a protein (or kinase) constitute vectors for the proteome (or phosphoproteome) level. A binary gene vector demonstrates which of the tumor and normal samples have a mutation in a specific gene (DNA biomarker).

In the present innovative solution dimensionality reduction is performed in step 526 by selecting only one biomarker from each cluster of the integrative biological network produced in step 523. This choice is done in order to avoid highly correlated features/biomarkers that increase complexity, and more importantly to avoid erroneously biasing outputs of the optimization algorithm (e.g. from using more potential biomarkers from a first cluster, as opposed to the fewer potential biomarkers of a smaller second cluster). The choice of a single biomarker per cluster is justified from the fact that due to their common function, members of the same cluster convey no or little additional information.

For each cluster, only the single molecule that provides the most informative description of the cluster, (e.g. the one that interacts with most of the cluster's members) is selected. With finding a representative molecule for each cluster, bias (resulting in false positives) is minimized and the search space reduces significantly making the algorithm faster. Alternatively, Spearman correlation can be computed between the vectors of each biomarker of a specific cluster. In this way, highly correlated biomarkers can be discarded.

Any optimization algorithm can be used in step 540 to find the optimal set of biomarkers. To optimize the biomarkers set, the search space of potential biomarkers is been explored and its solution is been assessed by an evaluation function which uses as parameters the patient's clinical data (e.g. blood pressure, cholesterol level, glucose level, medication, physiological signs, age, weight, diet, etc.) and associated clinical knowledge (e.g. high glucose level and high blood pressure are associated with a certain disease in patients over 60, taking a certain medication for a over a year, and for this disease a set of biomarkers are known to exist, where this set of biomarkers may is a subset of the set of biomarkers inputted to the optimization algorithm). The clinical data and associated knowledge are accessed from database 545. Algorithm 540 iterates until the quality threshold is exceeded (step 550) and a solution that performs well enough according to the quality threshold has been reached.

The optimization algorithm can be a multi-objective algorithm that can solve the problem of selecting the final biomarker sets and construct prediction models, which prediction models are able to classify samples to the different biological conditions with high accuracy. Vector machines and random forests are types of classifiers that may be used as prediction models. These classifiers take as input the vectors/features of the biomarkers. As defined above, these features define the value of the biomarkers for every available sample (disease and healthy samples). The classifier used is able to predict how well the features are able to distinguish disease and healthy samples collectively. This multi-objective algorithm initiates a population of solutions, which are represented as variables indicating whether a biomarker from the initial list should be selected or not.

By means of example, a genetic algorithm can be used for the optimization step 540 (A-B). This genetic algorithm in shown in FIG. 6.

In another exemplary embodiment, the multi-objective optimization method described in (540) can be substituted by any other optimization method (e.g. hill climbing method, Particle Swarm Optimization etc.) adding the restriction that two nodes in the same cluster of the integrative biological network should not be in the same subset of predictive biomarkers in order to avoid providing redundant inputs to the classification models deteriorating their accuracy and efficiency.

In yet another exemplary embodiment, the multi-objective optimization method is a Pareto-based method and uncovers a ranked list of equivalent Pareto-optimal biomarkers subsets with their related prediction models.

The quality metric of each solution i (where i represents a set of biomarkers that are used as input in a classifier) is given by Equation 1.

$$\frac{α * A U C ( i )}{β * ( \# biomarkers + \# trained models )} \qquad ( Equation 1 )$$

where AUC(i) reflects the accuracy of the classifier when the specific set of biomarkers of the solution i is used. AUC is the area under the curve that plots the true positive rate versus the false positive rate. The true positive rate is defined as (True Positives/(True Positives+False Negatives)) and the false positive rate as (False Positives/(False Positives+True Negatives)). The true positive rate defines the proportion of positives that are correctly identified as such and the false positive rate the proportion of positives that are incorrectly identified as such. In order to simplify the final model, we favor the solutions that use a limited number of biomarkers and have simplified trained models. To this end, we divide the AUC with the summation of the number of biomarkers and trained models. As an example, in the case of the support vector machine classifier, the number of trained models will be the number of support vectors that are used to distinguish disease from healthy samples. In order to avoid having extremely simple classifiers with low performance or extremely complicated classifiers with high performance, we use two parameters (α and β) which define the importance of each term in the quality metric. By varying these parameters, one can decide for the level of complexity of the final classifier.

Once optimization of the biomarker set has finished (B), the optimized biomarker set is annotated (step 560) with Gene Ontology (GO) terms from database 563 and molecular pathways from database 566. This annotation is done by identifying, in both the Gene Ontology terms and the molecular pathways, data associated with the optimized biomarkers.

Using the annotation of step 560, comparison is made between the final predicted biomarkers and known functional terms (such as GO terms or molecular pathways from databases like KEGG) to identify the affected cellular functions in the specific disease (step 570). This comparison is performed by comparing the set of biomarkers to every set of known biological function contained in the gene ontology terms and molecular pathways using the hypergeometric distribution to assess if the set of biomarkers is overrepresented in the set of the genes of each cellular function. Only those over-represented biomarkers above a threshold are selected.

The processing ends with reporting (step 580) the final biomarker set for the examined biological condition (e.g. a syndrome or a disease) together with the relevant prediction models and the affected cellular functions.

FIG. 6 shows the main steps of a genetic algorithm. Such an algorithm is a type of multi-objective algorithm used to optimize a set of solutions, where each of the solutions corresponds to a specific set of biomarkers resulted from genomics, transcriptomics, proteomics and other biological data.

The genetic algorithm starts (A) with step 610 where instances of the genetic algorithm are applied to the sets of potential biomarkers from all available omics and other biological data produced in step 540. A number of solutions 620, 623, 626 are produced and each of these solutions are evaluated in step 630. Instead of using a genetic algorithm, any other way of exploring the search space of the available solutions can be used (e.g. Monte Carlo approaches).

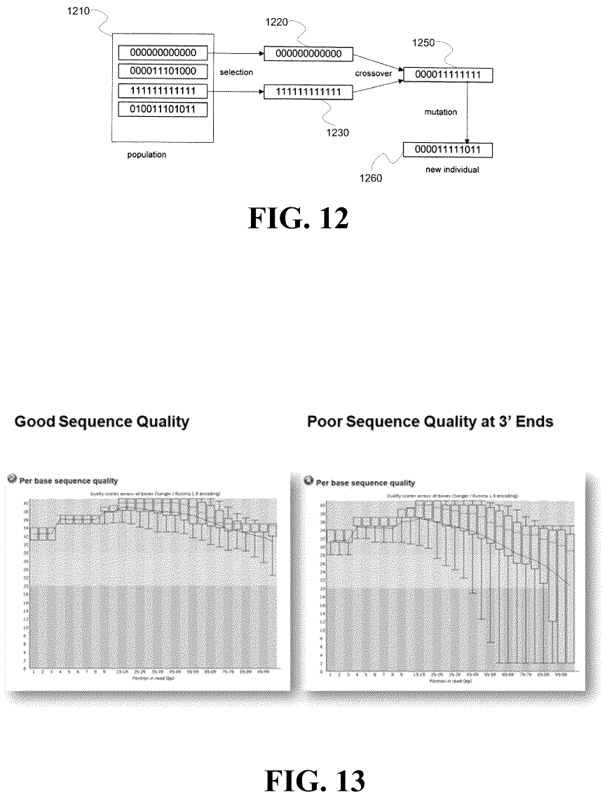

FIG. 12 shows an example of the application of the steps 640, 650. An initial population of biomarkers 1210 is represented as a sequence of “1” and “0” where “1” means to include the corresponding biomarker in the set and “0” means to discard it.

If a biomarker is chosen in the solution (“1”), this biomarker can correspond to many sources and/or features, such as RNA or proteome expression (also selected within the representation of the solution).

Two sets of biomarkers 1220, 1230 are selected (step 640). In this example, the two biomarker sets are arbitrarily selected so as to include no biomarker 1220, and to include all biomarkers 1230. In the variate step 650, a crossover step is applied to the two selected biomarker sets to produce a single crossover biomarker set 1250 consisting of a part of first biomarker set 1220 and a part of second biomarker set 1230. Parts of the first 1220 and the second 1230 biomarker sets are used in the crossover biomarker set 1250. The genetic algorithm continues by applying a mutation to the crossover biomarker set 1250 to create a new biomarker set 1260, which is evaluated in step 630.

The best performing solutions in the execution of the genetic algorithm have a higher chance to be selected in step 640, and variations of the parameters of the genetic algorithm are used in step 650 so as to allow the iterative application of the genetic algorithm on the candidate solutions until sufficiently good solutions are found judged by a quality metric against a quality threshold in step 660.

In an alternative exemplary embodiment, in addition or as a replacement to the performance metric, the number of iterations is used and once a user-defined maximum number of iterations is reached, the iterations terminate (B) and the optimized set of biomarkers is sent to step 560 for functional annotation.

Identifying Mutations as Biomarkers from DNA-Sequencing Data

The prevailing pipeline for identifying mutations as biomarkers from DNA-sequencing data consists of i) aligning the raw reads, which are generally formulated in FASTQ format to a reference genome stored in binary alignment map (BAM) files, and then ii) applying various variant calling algorithms to identify single nucleotide polymorphisms (SNPs), insertions, deletions and other genetic alterations. Such tools already exist. Some examples are GATK and SAMtools. The results of the variant calling algorithms are stored in a variant call file (VCF). Several algorithms exist for the different steps of this pipeline, while very few end-to-end pipelines and related tools exist. Moreover, computational methods have been proposed for the meta-analysis of the uncovered genetic variations in order to identify the ones that have impact at the protein level (non-synonymous) and those that are more likely to be disease-related. For the sake of this, gene annotation tools are used (e.g. SnpEff, VEP) to characterize the variants based on the genomic position and by assessing the functional impact of the corresponding amino acid substitution.

The proposed solution uses existing algorithms for DNA analysis and adds a functionality for selectively filtering predictions of deleterious SNPs, insertions and deletions.

FIG. 7 is a flowchart showing the main steps performed to predict biomarkers using DNA-Seq data. The processing starts with step 705 where the DNA-Seq Reads from database 707 are mapped to a Reference Genome, which reference genome is retrieved from database 703.

The input to step 705 is a set of sequencing data between two biological conditions resulted from a DNA-sequencing platform (e.g. healthy vs. disease samples). These sets of sequencing data are derived from biological experiments and the data are represented in a human-readable primary analysis output format called Sanger FASTQ, containing read identifiers, the sequence of bases, and the PHRED-like quality score Q, represented by single ASCII character to reduce the output file size.

Step 705 characterizes the experiments as having short, medium or long reads. Short reads are the ones of size less than 50 bases, medium reads are the ones with length between 50 and 100 bases and long reads are the ones with more than 100 bases. Then the reference genome is selected among a variety of available reference genomes with the default being the hg19 chromosome as provided by the Ensemble database. Then the actual mapping is realized in step 705 in order to generate a BAM/SAM file for each FASTQ input file. Sequence Alignment/Map (SAM) formatted files are files generated by read aligners containing sequences aligned to a reference sequence and other associated information. BAM files are losslessly compressed SAM files and the BAM files contain the comprehensive raw data of genome sequencing.

The DNA-Seq reads alignment in step 705 can be accomplished with any of the known aligners with the Bowtie-based or hash-based approaches being the default options. For these approaches, the parameters which should be used are the default ones (e.g. number of consequent allowed gaps, number of total gaps, etc.) for the type of reads (short, medium, long) of each dataset.

Step 710 then analyzes the genome coverage of the previously mapped DNA-Seq Reads from step 705 in order to perform quality control and discard poorly mapped samples. By means of example, the SAMtools are used in step 710. The output of step 710 is a set of Binary Alignment Map (BAM) and Sequence Alignment Map (SAM) files.

Variants in the BAM/SAM files are analyzed in step 715. Variant calling tools (such as SAMtools or any other similar algorithm or tool) are used to produce recalibrated Variant Call Files (VCF files). VCF files are text files storing gene sequence variations.

Taking for example the Read Sequences for a patient and the reference genome, a VCF file contains information on how these reads are aligned to the reference genome and how the genome of a patient is different from the reference genome (i.e. which variants of different types exist in the patient data).

Processing continues in step 717, where a selection is made (“1” or “2”) which determines if the filtering of variants based on their allele frequency is performed before (“1”) or after (“2”) the prediction of deleterious variants. A deleterious variant, or disease-causing variant is a genetic alteration that increases an individual's susceptibility or predisposition to a certain disease or disorder. When such a variant is present, development of the disease is more likely. This selection is made either manually by the user or automatically by software or hardware as presented in FIG. 9.

In this step, the variants described in the VCF files (which have been created in step 715) are filtered to keep the most significant variants. If mode “1” is selected, then the different variants are first filtered to identify deleterious variants. After that, the gene variants are filtered based on their occurrence in the available disease samples. For example, a gene is aberrant in at least 1% of the available disease samples (step 728). In the case of Single Nucleotide Variants (SNPs), these are filtered to keep only non-synonymous SNPs (step 721), meaning SNPs located in exons, which lead to amino acid changes in the protein sequence. Next step 722 filters and scores the SNPs according to other criteria, i.e. the functional impact of the change in the protein sequence. To predict the functional impact of the variants (SNPs, insertions or deletions), known classifiers are used (e.g. Mutation assessor and others). Alternatively, machine learning classifiers can be trained using data of known deleterious and neutral variants from publicly available repositories. In this case, the results of the tools for assessing the functional impact of the variants (Mutation assessor and others) are been used as input features for the machine learning classifier. The same analysis is done for insertions and deletions in steps 724, 726, respectively. The different predictors of deleterious SNPs (722), insertions (724) and deletions (726) are also extracting a confidence score for this variant being deleterious. Then, essentiality is (optionally) checked for all types of variants by multiplying the confidence score with a default constant value indicating that the variant is present in or absent from an essential gene. Essential genes are the genes for which normal functioning is vital for the survival of the cell they are located in.

Processing continues with the further filtering of mutations using the minimum allele (i.e. a variant form of a given gene) frequency threshold in Step 728 across the set of disease samples.

When mode “2” is selected, the minimum allele frequency threshold is applied first in step 738, prior to the other filters in steps 731, 732, 734, 736. The optimal mode of operation (i.e. “1” or “2”) is not known in advance and can be determined only after the application of the processing steps of FIG. 7. The mode of operation is optimized together with other parameters of the processing steps of FIG. 7. This optimization is presented in FIG. 9.

The output of mode “1” or mode “2” is a list of variants with their confidence scores. These variants from steps 720 and 730 are then statistically analyzed to assess if their occurrence in one biological condition (e.g. disease samples) is more prevalent compared to their occurrence in another biological condition (normal samples) in step 740. For the sake of this, a score is computed based on known statistical tests (chi square test) or tools (MutSigCV) in step 740. In cases where information of quantification is available in the form of copy numbers, other statistical tests such as student t-test or Wilcoxon Rank Sum test can be used to calculate a p-value for each variant comparing the mean or median of the copy number of each variation between the disease and normal samples. In principle, a mutation may happen in X numbers of DNA sequences in a sample and not happen in Y numbers of sequences in the same sample. The score is then compared with a predefined threshold in step 750 and it is above the threshold, it is discarded in step 760.

Copy number variation is a phenomenon in which sections of the genome are repeated and the number of repeats in the genome varies between individuals in the human population. Copy number variation is a type of structural variation, more specifically it is a type of duplication or deletion event that affects a considerable number of base pairs. Copy number variations play an important role in generating necessary variation in the population, as well as, in disease phenotypes.

The mutations identified in step 750 are ranked in step 770 with a confidence score which confidence score is the product of the confidence score calculated in steps 720, 730 and the value −log(p-value) calculated at step 740 for this mutation.

In an alternative exemplary embodiment, processing steps 700 take as input datasets of only one biological condition (e.g. disease samples). In this case, the variants are identified by comparing the disease samples to a reference genome.

In another exemplary embodiment, steps 720, 730 are implemented with a new ensemble feature selection methodology, which uses optimization algorithms (e.g. genetic algorithms and classification models (e.g. Support Vector Machines) to select an optimal subset of variants. The algorithm selects subsets of variants by heuristically searching different combinations in order to maximize the predictive accuracy (i.e. how well the algorithm differentiates the disease vs. the control samples) of the selected subset and by minimizing its size. Example algorithms that can be used as inputs include but are not limited to SIFT, PROVEAN, Polyphen, MutationAssessor, Oncodrive and iPAC. These example algorithms produce features (i.e. scores of the variants). These scores are used as features in any machine learning classifier to predict variants related to a specific disease.

Identifying Transcript Quantities as Biomarkers from RNA-Sequencing Data

The analysis of transcriptomics (i.e. RNA data) is mostly oriented towards the identification of biomarkers at the transcriptome for which relative expression levels are significantly differentiated between two biological conditions. This is usually accomplished with the use of RNA-Seq data. The prevailing pipelines for biomarker discovery using RNA-Seq data are designed for the identification of differentially expressed genes by comparing gene expression counts between two or more conditions. However, these pipelines are designed to be fully functional for identifying mRNAs and not short non-coding RNAs, such as miRNAs and tRNAs which are molecules that have been proven to play a significant role in gene regulatory mechanisms and carcinogenesis. Regarding short RNAs, there exist some tools and methods for parts of the analysis, such as the aligners PatMaN and MicroRazerS and the de novo identifiers of some specific categories of non-coding RNAs, such as miRDeep and ShortStack, but there does not exist a unique holistic pipeline for the discovery of short RNA biomarkers from transcriptomics data. In brief, these tools only predict a limited number of types of non-coding RNAs and their output is not linked to other important steps in RNA analysis, such as the differential expression analysis between different biological states. This problem is solved by the steps described in FIG. 8. FIG. 8 is a flowchart showing the main steps performed to identify biomarkers at the RNA level. The steps 800 in the flowchart use RNA-sequencing for discovering potential biomarkers with emphasis on non-coding RNA identification and include a mechanism for the integration of microarray experiments and network-based biomarkers.

The processing starts with inputting raw .FASTQ RNA-sequencing data files from database 807 and a reference genome or transcriptome selected among genome and transcriptome data stored in database 803. These data are quality controlled in step 805 and the processed .FASTQ data are fed to step 810.

The input data files are preprocessed in step 805 in order to remove the adapter sequence added to the reads by the sequencing platform. As an example, reads coming from Hi-seq sequencer are all having a specific sequence in the beginning (e.g., AAGGTTCA) which is the adapter sequence to be removed. Moreover, in order to identify biomarkers at the transcriptome level, the input dataset should have sufficient samples for each biological condition (e.g. more than two samples for control and more than two samples for disease state). The alignment can be implemented with any of the available algorithms and tools such as Tophat and Star.

In a variation of the present exemplary embodiment, the quality control part of step 895 includes demultiplexing. In some cases, molecular sequencing libraries are multiplexed into one pool of molecules and the sequencing may or may not perform the demultiplexing depending on its technology and the library preparation method. When data are multiplexed, and inline barcodes are part of the sequencing read, they are demultiplexed and the barcodes are removed from the reads.

In another embodiment, the quality control of step 805 comprises filtering and/or trimming reads by quality. Sequencing reads may contain sequencing errors. In order to avoid inserting such an error to the analysis, discarding and/or trimming reads is employed with criteria such as absolute minimum, average, and sliding-window-average quality scores.

An example quality score for each read position in the .fastq RNA-sequencing data files is shown in FIG. 13. In this example, the left image shows sequence with high quality, while the right image shows sequences with poor quality. For the right image all reads above position 75 are discarded due to poor quality by setting a corresponding threshold.

In yet another embodiment, none, some, all or other quality control checks are being employed at every possible order.

Step 810 aligns the processed .FASTQ data to the selected reference genome or transcriptome and produces a set of BAM and SAM files.

If the utilized dataset includes short reads, then processing continues in step 820 with [sub-step (i)] searching unaligned and/or aligned but unassigned in step 810 reads in non-coding RNA databases 823, such as miRBase or [sub-step (ii)] using in silico non-coding RNA predictors. A read can align to the reference transcriptome or genome or not align (i.e. aligned/unaligned). Afterwards, the aligned reads are used to infer the identified transcripts. However, for a transcript to be identified there need to be satisfied criteria such as minimum number of aligned reads, minimum number of uniquely aligned reads and so on. So, for some transcripts even if we have aligned reads they do not get identified. And these reads are aligned but not assigned. Unassigned reads are examined for differentiation, e.g. in different diseases since the unassigned reads can be implicated with the cause of the disease. Step 820 outputs a list of non-coding RNAs and their relative quantity per sample.

In a variation of the present exemplary embodiment, [sub-step (ii)] can be implemented prior to [sub-step (ii)].

Processing continues in step 825 (which is executed in parallel with step 820) where relative gene expression values of the assigned reads are calculated by using a publicly available genome annotation file and a method to read counts and taking into account the unassigned reads in BAM/SAM files of step 820, i.e. the format of the data when alignment to a genome has occurred.

Step 825 can be implemented with the Cuff tools or any other similar tool. Relative expression values of the transcripts provide information about the plurality in the samples. However, since the relative expression values are affected by the experimental design, the relative expression values are not the actual plurality of the transcripts in the samples but can only be used to compare late the transcripts with the pluralities of different transcripts in the same dataset.

Optionally, in case microarray experiments have been conducted for the same dataset, the microarray data from database 835 and the outputs from steps 820, 825 are fed to step 830. Microarray data are imaging data which are being preprocessed to get the quantities of transcripts in a sample.

Step 830 normalizes these three types of input data in order to homogenize RNA abundances from the two technologies (e.g. values initially ranging in RNA-seq from 0-100) to a single value window (by default [0, 1]).

An optional missing value imputation algorithm (added in step 830) is applied to all the normalized datasets in order to fill-in missing values (by default the k nearest neighbor imputation method is used).

Processing continues in step 840 by statistically analyzing differentially expressed genes at the RNA level to produce a 1^{st} set of biomarkers. The statistical analysis is done with the DESeq2 tool and a user-defined threshold (e.g. p-value 0.05, or False Discovery Rate 5%) to detect biomarkers as differentially expressed genes at the RNA level. Other statistical algorithms can be used in alternative exemplary embodiments.

In parallel with step 840, gene co-expression networks are created for each biological condition in step 850. These gene co-expression networks are compared to each other in step 855 (using InSyBio BioNets) to produce a 2^{nd} set of biomarkers.

In another exemplary embodiment, the gene co-expression networks are combined with physical Protein-Protein Interaction Networks (PPIN). This combination can be done by filtering out edges from the co-expression networks that do not exist in the protein-protein interaction networks, therefore reducing the dimensionality of the problem resulting in faster execution and minimizing bias (false positives) from the eliminated edges.

The 1^{st} and 2^{nd} set of biomarkers from steps 840 and 855, respectively, are fed to step 860. Step 860 combines the differentially expressed biomarkers of the 1^{st} biomarker set with the network-based biomarkers of the 2^{nd} biomarker set. A confidence score is then calculated in step 880 for the combined biomarkers.

Step 860 can be implemented with InSyBio BioNets or a similar tool. In InSyBio BioNets this combination is conducted by computing a new confidence score which is the average of (l-pvalue) which we get from the differential expression analysis and of the confidence score which is the output from the network comparison methods.

In another embodiment, the non-coding RNA biomarkers which act as regulatory molecules, such as microRNAs and transfer-RNAs, is further filtered by keeping only the ones that produce relevant results in association with their targeted genes. In specific, a target prediction tool may be used to identify genes that are regulated by a non-coding RNA. It is known, for example, that miRNAs target genes and reduce their quantity. Accordingly, it is expected that targets of increased quantity miRNAs will exhibit decreased quantity. Else, we consider that the miRNA-target interaction is not active in the specific dataset.

Processing continues in step 870 by ranking the combined biomarkers according to the calculated confidence scores and the processing ends with step 890 by reporting the ranked biomarkers.

Identifying Transcript Quantities as Biomarkers from Proteomics Data

Proteomics data are being produced by analyzing bio fluids or samples from tissues using Mass Spectrometry based experimental instrumentation. The raw data emerging from these types of experiments consist of thousands of spectral graphs with each spectral graph corresponding to a peptide, where a peptide is defined as a fragment of a protein. The standard analysis of these data start from preprocessing spectral graphs to remove noise, detect and filter peaks. The next step is to search these spectral graphs against a protein set of interest (e.g the Uniprot Human Proteome) using computational commercial (e.g. Mascot) or open source tools (e.g. Xtandem). With this search peptides and proteins are identified. The next step is the quantification of proteins to detect the relative quantity of each protein in the sample, using the precursor masses in label-free proteomics technologies or the quantification peaks in labeled proteomics. In another embodiment, the “Quantify then Identify” technique used in InSyBio's QtI Tool can be applied to perform a first quantification and then identification so that more quantified spectra and proteins can be detected from the same experiment. When the relative quantities of the proteins are measured, the analysis is the same as in transcriptomics data (FIG. 8, steps 840-870) including differential expression analysis and biological network comparison to locate and identify biomarkers.

Automated Optimization of Biomarker Discovery Algorithms for Diseases/Medical Conditions

An additional drawback of existing computational pipelines for the discovery of molecular biomarkers is that most of them use different algorithmic solutions, which require tuning various parameters. The selection of the suitable algorithms and the optimal parameters is a time-consuming procedure, which deters non-bioinformatics experts from using such a solution. Moreover, the default algorithms and parameters described in each approach are mostly appropriate for a specific dataset and cannot be generalized to other datasets and diseases. These problems are solved by the innovative solution presented in the steps of FIG. 9.

FIG. 9 is a flowchart showing the main steps performed to automate the optimization of biomarker discovery algorithms for diseases and medical conditions. Steps 900 can be used in the problem of detecting biomarkers for diseases as well as for other tasks such as personalized nutrition. Steps 900 are applied to identify the optimal algorithmic mix, order and parameters based on the present innovative solution for specific fields, such as cancer, neurodegenerative diseases and nutrition.

Processing commences with task 910 which inputs disease-related metadata such as DNA-sequencing, transcriptomics and proteomics data, experimentally verified biomarkers from database 906, and clinical data such as cholesterol levels, blood sugar levels, imaging-related variables for neurodegenerative diseases, and medication from database 903 (e.g. a doctor's or hospital database, or a patient's medical folder). These variables are used in the feature selection algorithms.

Step 920 randomly initializes the algorithmic steps shown in FIG. 3 and step 930 applies the randomly initialized algorithms to the input data in step 910 and produces a vector of variables of algorithm sets.

Then, an initial population of solutions is generated in step 940. Each solution is an instance of the biomarker discovery method presented in FIG. 3. Each solution is been represented in a vector of variables which show the selection of every algorithm (among a predefined set of potential algorithms to be used) and the selection of each parameter. Moreover, the representation scheme allows each solution to represent whether the method for the analysis of DNA-sequencing experiments should be used in mode 1 or 2 (step 717). In addition, the solution is able to select or discard any part of the pipelines described in FIG. 7-8. For example, in FIG. 7 the variants can be filtered or not based on the variant allele frequency (steps 728, 738). Moreover, the solution is able to vary the parameters used in the pipeline and choose the optimal values during the procedure of the optimization. These parameters include the thresholds at steps 717, 728, 738 and 750.

The processing continues with step 950 where the standard steps of a genetic algorithm are applied (refer to FIG. 6) until some solution with sufficiently high performance is found.

The evaluation of the different solutions of the genetic algorithm of step 950 is conducted by executing the genetic algorithm for each solution using the representative biological datasets for this biological/medical problem and calculating the following metrics: ability of the pipeline to propose biomarkers that better distinguish disease and normal samples (assessed by the AUC metric), average time and memory requirements for running the overall pipeline. The latter two goals are minimized, while the prediction metrics are maximized.

The method depicted in FIG. 9 leads to obtaining the default method (algorithms and parameters selected) for each field of interest. Example fields are cancer, neurodegenerative diseases and nutrition.

FIG. 10 shows an example of an integrative biological network. The network maps genes, mRNA and proteins onto nodes and connects nodes interacting with each other using edges. The edge thickness represents a weight associated with each edge and is associated with a metric like confidence on the association, degree of association etc. The integrative network of FIG. 10 is constructed using Transcriptomics and Proteomics analysis data and associated knowledge from scientific databases and analysis tools like Uniprot, miRTarget, InSyBio ncRNAseq and InSyBio Interact.

FIG. 11 shows an example of a clustered integrative biological network. The GENA clustering algorithm has been applied to the integrative biological network of FIG. 10 to predict the clusters 1110. After the application of the clustering algorithm, a number of unclustered molecules still remain (EIF3CL, Protein10, Protein11, Protein1_Glycolysis_PTM, mRNA4, mRNA5, mRNA6, tRF1).

Below the clustered biological molecules 1110 are shown the Equivalent Disease Predictive Models uncovered from the biological clustering driven dimensionality reduction using the Hybrid Genetic Algorithms-SVM ensemble technique 1120.

The above exemplary embodiments are intended for use either as a standalone user identification method in any conceivable scientific and business domain, or as part of other scientific and business methods, processes and systems.

The above exemplary embodiment descriptions are simplified and do not include hardware and software elements that are used in the embodiments but are not part of the current invention, are not needed for the understanding of the embodiments, and are obvious to any user of ordinary skill in related art. Furthermore, variations of the described method, system architecture, and software architecture are possible, where, for instance, method steps, and hardware and software elements may be rearranged, omitted, or added.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment and best mode of the invention known to the applicant at this time of filing the application has been presented and is intended for the purposes of illustration and description. It is not intended to be exhaustive or limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out this invention, but that the invention will include all embodiments falling within the scope of the appended claims.

In one or more exemplary embodiments, the functions described may be implemented in hardware, software, firmware, or any combination thereof. If implemented in software, the functions may be stored on or transmitted over as one or more instructions or code on a computer readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer or any other device or apparatus operating as a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. In an information handling system, a method of computational prediction of biomarkers, associated with a biological condition, in omics data, comprising:
analyzing omics data to predict a first set of biomarkers for each type of omics data;
constructing a first biological network, where the first biological network maps different types of omics data onto nodes and connects the nodes with edges by exploiting overlapping information from individual biological networks, and where each of each individual biological network maps only one type of omics data;
clustering the first biological network into clusters of biomarkers of biological significance;
creating a second set of biomarkers by selecting from each cluster a single biomarker that conveys the most information about the cluster;
creating a third set of biomarkers by applying an optimization algorithm to the third set of biomarkers and using clinical data and associated clinical knowledge as parameters to the optimization algorithm;
annotating the third set of biomarkers with gene ontology terms and molecular pathways by identifying, in both the gene ontology terms and the molecular pathways, data associated with the optimized biomarkers; and
identifying cellular functions of the biological condition by comparing the third set of biomarkers with cellular functions from the gene ontology and molecular pathways, where the functionalities are affected by the third set of biomarkers.

2. The method of claim 1, where the creation of the second set of biomarkers is done by selecting from each cluster the biomarker that interacts with most of the cluster's members.

3. The method of claim 1, where the creation of the second set of biomarkers is done by computing the Spearman correlation between a vector of each biomarker of a specific cluster and discarding highly correlated biomarkers until only one biomarker is left in each cluster, and where the vector of each biomarker has a length equal to the number of data samples and the vector comprises relative expression measurements for each of the samples or a binary vector that indicates the presence of a variation in the sample or a clinical variable.

4. The method of claim 1, where the optimization algorithm comprises a genetic algorithm or a multi-objective algorithm

5. The method of claim 1, where the cellular functions of the biological condition are identified by comparing the third set of biomarkers to every set of known biological function contained in the gene ontology terms and molecular pathways using the hypergeometric distribution to assess if the set of biomarkers is over-represented in the set of the genes of each cellular function and selecting those only over-represented biomarkers that are above a threshold.

6. The method of claim 1, further comprising:
randomly initializing the selection of algorithms for the steps of method 1, the order of execution of the algorithms and the parameters of the algorithms;
optimizing the outputs of the randomly initialized algorithms; and
reporting the optimum selection of algorithms, the optimum order of execution of the algorithms and the optimum parameters of the algorithms.

7. The method of claim 1, where the omics data comprise genomics, transcriptomics, proteomics data.

8. The method of claim 1, where the omics data that are analyzed are genomics data, the method further comprising:
mapping DNA sequence reads to a reference genome;
analyzing genome coverage;
analyzing variants in the DNA sequence reads;
predicting deleterious variants and filtering the predicted deleterious variants by comparing the predicted deleterious variants against an allele frequency threshold;
keeping only variants that are more representative in the population of disease samples compared to normal samples; and
ranking variants according to a confidence score.

9. The method of claim 8, where the allele frequency threshold is used to filter the analyzed variants in the DNA sequence reads prior to predicting deleterious variants, instead of filtering the predicting deleterious variants.

10. The method of claim 1, where the omics data that are analyzed are transcriptomics data, the method further comprising:
preprocessing RNA sequencing data;
aligning the preprocessed RNA sequencing data to a reference genome or transcriptome;
calculating relative gene expression values for the aligned RNA reads;
finding unassigned unaligned short RNA reads and unassigned aligned short RNA reads by querying non-coding RNA databases or applying a prediction algorithm to the RNA sequencing data;
identifying differentially expressed genes in unassigned RNA reads between diseases and normal samples;
normalizing and combining aligned RNA reads, unaligned RNA reads, and microarray data;
statistically analyzing the differentially expressed genes to create a first set of biomarkers;
creating gene co-expression networks for each biological condition using the combined aligned RNA reads, unaligned RNA reads, and microarray data;
comparing gene co-expression networks to create a second set of biomarkers;
combining the first and second set of biomarkers; and
ranking the combined set of biomarkers using a confidence score.

11. An information processing system configured to computationally predict biomarkers in omics data, where the biomarkers are associated with a biological condition, comprising:
means for analyzing omics data to predict a first set of biomarkers for each type of omics data;
means for constructing a first biological network, where the first biological network maps different types of omics data onto nodes and connects the nodes with edges by exploiting overlapping information from individual biological networks, and where each of each individual biological network maps only one type of omics data;
means for clustering the first biological network into clusters of biomarkers of biological significance;
means for creating a second set of biomarkers by selecting from each cluster a single biomarker that conveys the most information about the cluster;
means for creating a third set of biomarkers by applying an optimization algorithm to the third set of biomarkers and using clinical data and associated clinical knowledge as parameters to the optimization algorithm;
means for annotating the third set of biomarkers with gene ontology terms and molecular pathways by identifying, in both the gene ontology terms and the molecular pathways, data associated with the optimized biomarkers; and
means for identifying cellular functions of the biological condition by comparing the third set of biomarkers with cellular functions from the gene ontology and molecular pathways, where the functionalities are affected by the third set of biomarkers.

12. The information processing system of claim 11, further comprising:
means for randomly initializing the selection of algorithms for the steps of method 1, the order of execution of the algorithms and the parameters of the algorithms;
means for optimizing the outputs of the randomly initialized algorithms; and
means for reporting the optimum selection of algorithms, the optimum order of execution of the algorithms and the optimum parameters of the algorithms.

13. The information processing system of claim 11, where the creation of the second set of biomarkers is done by selecting from each cluster the biomarker that interacts with most of the cluster's members.

14. A non-transitory computer program product that causes an information processing system to computationally predict biomarkers in omics data, where the biomarkers are associated with a biological condition, the non-transitory computer program product having instructions to:
analyze omics data to predict a first set of biomarkers for each type of omics data;
construct a first biological network, where the first biological network maps different types of omics data onto nodes and connects the nodes with edges by exploiting overlapping information from individual biological networks, and where each of each individual biological network maps only one type of omics data;
cluster the first biological network into clusters of biomarkers of biological significance;
create a second set of biomarkers by selecting from each cluster a single biomarker that conveys the most information about the cluster;
create a third set of biomarkers by applying an optimization algorithm to the third set of biomarkers and using clinical data and associated clinical knowledge as parameters to the optimization algorithm;
annotate the third set of biomarkers with gene ontology terms and molecular pathways by identifying, in both the gene ontology terms and the molecular pathways, data associated with the optimized biomarkers; and
identify cellular functions of the biological condition by comparing the third set of biomarkers with cellular functions from the gene ontology and molecular pathways, where the functionalities are affected by the third set of biomarkers.

15. The non-transitory computer program product of claim 15 having further instructions to:
randomly initialize the selection of algorithms for the steps of method 1, the order of execution of the algorithms and the parameters of the algorithms;
optimize the outputs of the randomly initialized algorithms; and
report the optimum selection of algorithms, the optimum order of execution of the algorithms and the optimum parameters of the algorithms.

16. The non-transitory computer program product of claim 15, where the creation of the second set of biomarkers is done by selecting from each cluster the biomarker that interacts with most of the cluster's members.
